(19) United States
(12) Patent Application Publication
(10) Pub. No.: US 2013/0231753 A1
(43) Pub. Date: Sep. 5, 2013

(54) ENDOLUMINAL PROSTHESIS HAVING ANTI-MIGRATION COATING

(71) Applicant: COOK MEDICAL TECHNOLOGIES LLC, Bloomington, IN (US)

(72) Inventors: Alison Liddy, Galway (IE); Tríona Campbell, Co Clare (IE)

(73) Assignee: COOK MEDICAL TECHNOLOGIES LLC, Bloomington, IN (US)

(21) Appl. No.: 13/778,972

(22) Filed: Feb. 27, 2013

Related U.S. Application Data
(60) Provisional application No. 61/605,949, filed on Mar. 2, 2012.

(51) Int. Cl. A61F 2/82

(52) U.S. Cl. CPC A61F 2/82
USPC 623/23.7

(57) ABSTRACT

A prosthesis for placement within a body vessel of a patient may include a tubular frame and a covering material coupled to the tubular frame. The covering material may have an outer surface defining at least a segment of an outer surface of the prosthesis. An anti-migration coating may be applied to at least a portion of the outer surface of the prosthesis. The anti-migration coating may include a mucoadhesive agent. The mucoadhesive agent may include a polymer configured to adhere to a mucous membrane within the body vessel. The anti-migration coating may have a thickness of greater than or equal to about 2 μm in a dry state to promote adhesion to the mucous membrane.

### CROSS-REFERENCE TO RELATED APPLICATIONS

This application claims priority and the benefit of provisional U.S. Patent Application Ser. No. 61/605,949, filed Mar. 2, 2012, which is incorporated herein by reference in its entirety.

### TECHNICAL FIELD

This disclosure relates generally to medical devices and methods of use. More particularly, it relates to an endoluminal prosthesis having an anti-migration coating.

### BACKGROUND

Prostheses such as self-expanding metal stents may be implanted within a body vessel of a human or animal body for any one of a number of different purposes. For example, an esophageal stent may be implanted for treatment of dysphagia, fistula, anastomatic leaks, or benign esophageal strictures. Such stents may be bare stents, covered stents, or partially covered stents. Non-metallic prostheses such as plastic or silicone prostheses may be used instead of, or in addition to, self-expanding metal stents.

A portion of the esophagus may be compromised by the presence of a cancerous tumor. Such a tumor commonly may be found in the vicinity of the lower esophageal sphincter. The cancerous tumor growth typically impinges the flow of food and fluids through the esophagus. Lower esophageal cancer in the United States may affect as many as 12,000 patients per year. The incidence in the United States is approximately 5.1 per 100,000 people, and is rising, particularly in white male patients. Esophageal prostheses or stents are typically utilized in these cancerous patients.

In these patients, an esophageal prosthesis or stent typically is placed in the esophagus near the cancerous tumor to maintain the patency of the esophagus. The prosthesis may include one or more bare stents, partially covered stents, or covered stents. Typically, the stents are self expanding metal stents so that the prosthesis will expand upon implantation within the esophagus without further manipulation by the physician.

It has been reported that, on average, about 20% of covered stents migrate within the body vessel after implantation. Migration of an esophageal prosthesis may result in a failure of the prosthesis to maintain the patency of the esophagus and total or partial blockage of the esophagus by the cancerous tumor.

In recent years, much progress has been achieved in the development of materials that adhere or stick to the mucous membranes of the body. Such mucoadhesive materials are materials designed to adhere to a mucous membrane and have been incorporated into pharmaceuticals for localized drug delivery to locations within the body near the mucous membrane (e.g., the body tissue underlying the mucous membrane). It has been proposed to provide a pharmaceutical formulation incorporating a mucoadhesive and one or more bioactive agents. The mucoadhesive agent may retain the bioactive agent in contact with the mucous membrane to deliver the bioactive agent to the desired location within the body.

It may be desirable to incorporate such a mucoadhesive agent into an anti-migration coating to fix a prosthesis in place at a specific location within the body.

### SUMMARY

The present embodiments provide an endoluminal prosthesis having an anti-migration coating.

In one example, a prosthesis for placement within a body vessel of a patient may include a tubular frame and a covering material coupled to the tubular frame. The covering material may have an outer surface defining at least a segment of an outer surface of the prosthesis. An anti-migration coating may be applied to at least a portion of the outer surface of the prosthesis. The anti-migration coating may include a mucoadhesive agent. The mucoadhesive agent may include a polymer configured to adhere to a mucous membrane within the body vessel. The anti-migration coating may have a thickness of greater than or equal to about 2 μm in a dry state to promote adherence to the mucous membrane.

In another example, a prosthesis for placement within a body vessel of a patient may include a tubular frame having a compressed configuration and an expanded configuration. The prosthesis may include a first contact area positioned near a proximal end of the prosthesis and a second contact area positioned near a distal end of the prosthesis. The prosthesis may include an anti-migration coating disposed on at least a portion of an outer surface of the prosthesis. The anti-migration coating may include a mucoadhesive agent. The mucoadhesive agent may include a polymer configured to adhere to a mucous membrane within the body vessel. The polymer may have a molecular weight of less than or equal to about 200,000. With the tubular frame in the expanded configuration within the body vessel, at least a portion of each of the first and second contact areas may engage a wall of the body vessel to retain the prosthesis in place relative to the body vessel.

In another example, a method for making a prosthesis for placement within a body vessel of a patient may include providing a tubular frame and coupling a covering material to the tubular frame. The covering material may have an outer surface. The method may include applying an anti-migration coating having a thickness of greater than or equal to about 2 μm in a dry state to at least a portion of the outer surface of the covering material. The anti-migration coating may include a mucoadhesive agent. The mucoadhesive agent may include a polymer configured to adhere to a mucous membrane within the body vessel.

In another example, a method of treating an esophagus may include introducing an expandable prosthesis into the esophagus in a compressed configuration. The method may include expanding the prosthesis from the compressed configuration such that the prosthesis engages a wall of the esophagus. The prosthesis may include a mucoadhesive agent disposed on an outer surface of prosthesis. The mucoadhesive agent may include a polymer configured to adhere to a mucous membrane disposed on the wall of the esophagus. The method may include adhering the prosthesis to the mucous membrane of the esophagus with the mucoadhesive agent to fix the prosthesis in place relative to the esophagus. Adhering the prosthesis to the mucous membrane may include penetrating the mucous membrane with the polymer of the mucoadhesive agent.

Other systems, methods, features, and advantages of the invention will be, or will become, apparent to one with skill in the art upon examination of the following figures and detailed description. It is intended that all such additional systems, methods, features, and advantages be within the scope of the invention, and be encompassed by the following claims.

### BRIEF DESCRIPTIONS OF THE DRAWINGS

FIG. 1 depicts one embodiment of an endoluminal prosthesis.

FIG. 2 depicts another embodiment of an endoluminal prosthesis.

FIG. 3 depicts an esophageal prosthesis disposed within a cancerous portion of an esophagus.

FIG. 4 depicts an endoluminal prosthesis having an anti-migration coating applied to a surface thereof in one exemplary configuration.

FIG. 5 depicts an endoluminal prosthesis having an anti-migration coating applied to a portion of a surface thereof in one exemplary configuration.

FIG. 6 depicts one embodiment of a polymer chain arrangement on a surface of an endoluminal prosthesis.

FIG. 7 depicts another embodiment of a polymer chain arrangement on a surface of an endoluminal prosthesis.

### DETAILED DESCRIPTION OF THE DRAWINGS AND THE PRESENTLY PREFERRED EMBODIMENTS

For the purposes of promoting an understanding of the principles of the present disclosure, reference will now be made to the embodiments illustrated in the drawings, and specific language will be used to describe the same.

In the following discussion, the terms “proximal” and “distal” will be used to describe the opposing axial ends of a medical device, as well as the axial ends of various component features. The term “proximal” is used in its conventional sense to refer to the end of the device (or component thereof) that is closest to the physician during a medical procedure. The term “distal” is used in its conventional sense to refer to the end of the device (or component thereof) that is initially inserted into the patient, or that is closest to the patient during use.

Generally speaking, the present disclosure is directed to an endoluminal prosthesis for implantation within a human or animal body. The prosthesis includes an anti-migration coating to retain the prosthesis in place relative to a body vessel in which the prosthesis may be placed.

Although the following description will generally describe a prosthesis for implantation in the esophagus, the disclosure is not so limited. A prosthesis generally as described below may be configured for implantation in any vessel or cavity of a human or animal body. A prosthesis having an anti-migration coating may be particularly useful for implantation in a body vessel or cavity having a mucosal lining such as, for example, a urethra, a fallopian tube, a buccal cavity, a vagina, a nasal cavity, a vessel or cavity of a gastrointestinal tract, or a vessel or cavity of a cardiovascular system. Additionally, although the following description will generally describe a covered stent, the disclosure is not so limited. An anti-migration coating as described below may be applied to any type of prosthesis for implantation in a body vessel. For example, an anti-migration coating may be applied to a valve configured for placement in the sphincter to fix the valve in place relative to the sphincter muscle.

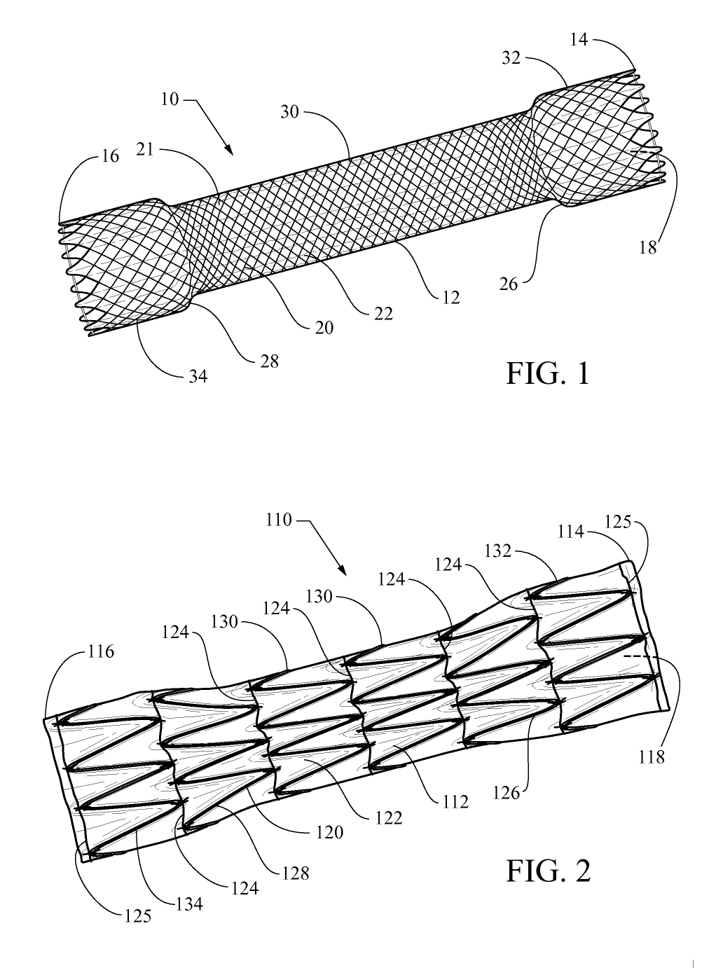

FIG. 1 illustrates one embodiment of an endoluminal prosthesis 10. The prosthesis 10 may be configured for implantation within an esophagus of a patient as further described below. The prosthesis 10 may include a tubular member 12. The tubular member 12 may have a first end 14, a second end 16, and a lumen 18 extending generally longitudinally within the tubular member between the first and second ends thereof. The lumen 18 may be configured to allow flow of a fluid or other material through the prosthesis 10.

The tubular member 12 of the prosthesis 10 may be defined by a generally tubular frame including one or more stents 20. In one example, the tubular frame may be configured as a conventional closed cell structure 21 as shown in FIG. 1. It should be noted that the illustrative stent configuration is merely exemplary, and it is contemplated that other stents and stent configurations may be substituted for the illustrative stent frame. The tubular frame may be made from a woven wire structure, a laser-cut cannula, individual connected rings, or any other type of stent structure known in the art. The closed cell structure 21 may be self-expanding or balloon expandable. Preferably, the closed cell structure 21 is self-expanding.

At least a portion of the tubular frame may be covered by a covering material. The covering material may be configured as a sleeve 22 that may be disposed around the frame. The sleeve 22 may be positioned on an outer surface of the tubular frame or an inner surface of the tubular frame (i.e., within the lumen 18). In other examples, the tubular frame may be disposed within layers of the sleeve 22, for example, using lamination or other known techniques. Preferably, the sleeve 22 may be positioned on the outer surface of the tubular frame to form an exterior surface of the prosthesis 10. The sleeve 22 may extend along substantially an entire length of the prosthesis 10 between the first end 14 and the second end 16 of the tubular member 12. The tubular frame and the sleeve 22 may cooperatively form a covered prosthesis.

In other examples, the covering material may cover only a portion of the tubular frame. For example, the covering material may be configured as a patch or sheet of material, which may partially extend around the circumference of the tubular frame to cover a portion of the circumference of the tubular frame. Additionally, or alternatively, the covering material may partially extend along the length of the tubular frame to cover a portion of the length of the tubular frame. In these examples, the covering material may be attached to the tubular frame such that the tubular frame and the covering material may cooperatively form a partially covered prosthesis.

The tubular frame may include first and second cylindrical end portions 32, 34 positioned near the first end 14 and the second end 16, respectively, of the prosthesis 10. The first and second cylindrical end portions 32, 34 may generally define proximal and distal ends, respectively, of the prosthesis 10. First and second flared portions 26, 28 may be positioned between the first and second cylindrical end portions 32, 34. For example, the first flared portion 26 may be positioned adjacent the first cylindrical end portion 32, and the second flared portion 28 may be positioned adjacent the second cylindrical end portion 34. A cylindrical middle portion 30 may be positioned between the flared portions 26, 28. The flared portions 26, 28 and the cylindrical end portions 32, 34 may aid in fixing the prosthesis 10 in place relative to the body vessel. For example, the cylindrical end portions 32, 34 may be configured as contact areas intended to engage the wall of the body vessel upon implantation of the prosthesis as further described below. In any of the examples described herein, the cylindrical portions (e.g., the cylindrical end portions and/or the cylindrical middle portions) may have any suitable cross sectional shape (e.g., elliptical, rectangular, triangular, or any other polygonal or nonpolygonal shape).

By way of example, the flared portions 26, 28 may have a minimum diameter of about 15 mm and a flared diameter of up to about 30 mm. The cylindrical middle portion 30 may have a nominal diameter of about 15 mm to correspond to the minimum diameter of the flared portions 26, 28. The cylindrical end portions 32, 34 may have a nominal diameter of up to about 30 mm to correspond to the flared diameter of the flared portions 26, 28. These diameters are nominal diameters for the tubular frame and may be customized to meet the particular demands of any human or veterinary patient. For example, a tubular frame having smaller diameters may be used to treat pediatric patients. Also for example, a tubular frame having smaller diameters may be suitable for use in locations such as a urethra, a fallopian tube, or a cardiovascular system.

FIG. 2 illustrates another embodiment of an endoluminal prosthesis 110. The prosthesis 110 may include a tubular member 112 having a first end 114, a second end 116, and a lumen 118 extending generally longitudinally within the tubular member between the first and second ends thereof. The tubular member 112 of the prosthesis 110 may be defined by a generally tubular frame including one or more stents 120. In this embodiment, the tubular frame of the prosthesis 110 may include a plurality of conventional zig-zag wire stents 120. The tubular frame may be generally covered by a sleeve 122 that may be disposed around the frame as described above with reference to FIG. 1.

The tubular frame may include a plurality of self-expanding stents 120 which may be interconnected by one or more filaments 124. For example, two adjacent stents 120 of the tubular frame may be positioned in an end-to-end relationship with one another. The filament 124 may extend generally circumferentially around the tubular frame and engage the adjacent ends of the two adjacent stents 120 to couple the adjacent stents to one another. The filaments 124 used to couple adjacent stents 120 to one another may be formed from any suitable filamentary material. For example, the filaments 124 may be formed from 3/0 diameter mononylon suture material. The interconnecting filaments 124 may maintain the diameters of the stents. In other words, the interconnecting filaments 124 may be configured to limit the expansion of any of the stents to a predefined diameter. Additionally, or alternatively, end filaments 125 may be positioned at the ends of the cylindrical end stents 132, 134 corresponding to the first end 114 and the second end 116, respectively, of the prosthesis 110 to maintain the diameters of the cylindrical end stents.

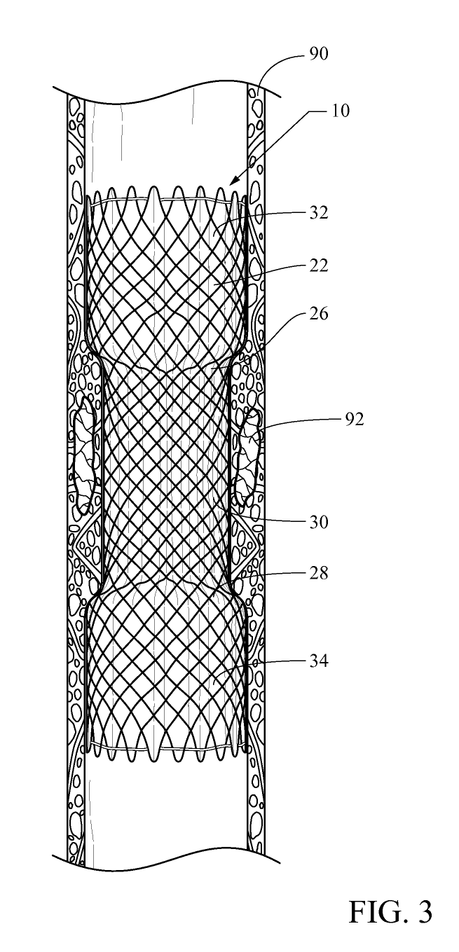

In the illustrative embodiment of FIG. 2, the tubular frame includes six self-expanding zig-zag wire metal stents, which may be of the Gianturco type as described in U.S. Pat. No. 4,580,568 to Gianturco, which is incorporated by reference herein in its entirety. The tubular frame may include first and second cylindrical end stents 132, 134 positioned near the first end 114 and the second end 116, respectively, of the prosthesis 110. The first and second cylindrical end stents 132, 134 may generally define proximal and distal ends, respectively, of the prosthesis 110. First and second flared stents 126, 128 may be positioned between the first and second cylindrical end stents 132, 134. For example, the first flared stent 126 may be positioned adjacent the first cylindrical end stent 132, and the second flared stent 128 may be positioned adjacent the second cylindrical end stent 134. One or more cylindrical middle stents 130 may be positioned between the flared stents 126, 128. The flared stents 126, 128 and the cylindrical end stents 132, 134 may aid in fixing the prosthesis 110 in place relative to the body vessel as further described below. Although FIGS. 3-9 are described below with reference to the prosthesis 10 shown in FIG. 1, the discussion is equally applicable to the prosthesis 110 shown in FIG. 2.

In any of the examples described herein, the tubular frame of a prosthesis may include any suitable materials such as, for example, stainless steel, nitinol, cobalt-chrome alloys, amorphous metals, tantalum, platinum, gold, or titanium. Alternatively, or additionally, the tubular frame may include non-metallic materials such as thermoplastics or other polymers.

The covering material, such as the sleeve, of a prosthesis may be formed from any conventional material. Preferably, the covering material may be formed from a liquid impermeable material that will not degrade in the presence of fluids or other gastric materials that may contact the prosthesis after implantation within the body vessel. For example, the covering material may be formed from silicone, polyurethane, nylon, polyamide (including other urethanes), or any other suitable biocompatible material. Additionally, or alternatively, the covering material may be configured as a polymer membrane.

The covering material may be formed using any conventional technique. In one example, the tubular frame of the prosthesis 10 may be mounted on a mandrel and dipped into a slurry material to cover the tubular frame with a layer of the slurry material. The slurry material may include any of the materials described above that may be used to form the covering material. The covered tubular frame may be dipped into the slurry material multiple times to increase the thickness of the layer of slurry material on the tubular frame (e.g., to dispose multiple layers of slurry material on the tubular frame). The slurry material on the covered tubular frame may be cured to form the sleeve 22. In other examples, the sleeve may be formed independently of the tubular frame and attached to the tubular frame. In still other examples, the covering material may be formed as a sheet or patch of material which may cover at least a portion of the tubular frame. The covering material may be attached to the tubular frame in any manner known in the art to form a covered or partially covered prosthesis.

FIG. 3 depicts the prosthesis 10 deployed within an esophagus to bridge a cancerous portion of the esophagus. The prosthesis 10 may be deployed within the esophagus using any suitable delivery technique known in the art. For example, the prosthesis 10 may be compressed into a reduced diameter delivery configuration and loaded into a deployment system. The prosthesis 10 may be deployed using any suitable deployment system. One exemplary deployment system is shown in U.S. Patent Application Pub. No. 2009/0281610 by Parker, which is incorporated by reference herein in its entirety. The prosthesis 10 in the deployment system may be advanced to the desired site of placement within the esophagus. The prosthesis 10 may be deployed from the deployment system and allowed to expand to an expanded configuration within the esophagus. Upon expanding, a portion of the prosthesis 10 (e.g., the sleeve 22) may engage the esophageal wall 90 as shown in FIG. 3. The cylindrical end portion 32 and the flared portion 26 may be positioned substantially proximal of a cancerous tumor 92 while the cylindrical end portion 34 and the flared portion 28 may be positioned substantially distal of the cancerous tumor. The cylindrical middle portion 30 may substantially bridge the cancerous tumor 92. The flared portions 26, 28 and the larger diameter cylindrical end portions 32, 34 may engage the esophageal wall 90 to reduce the probability of migration of the prosthesis 10 within the esophagus by exerting a radial outward force against the esophageal wall.

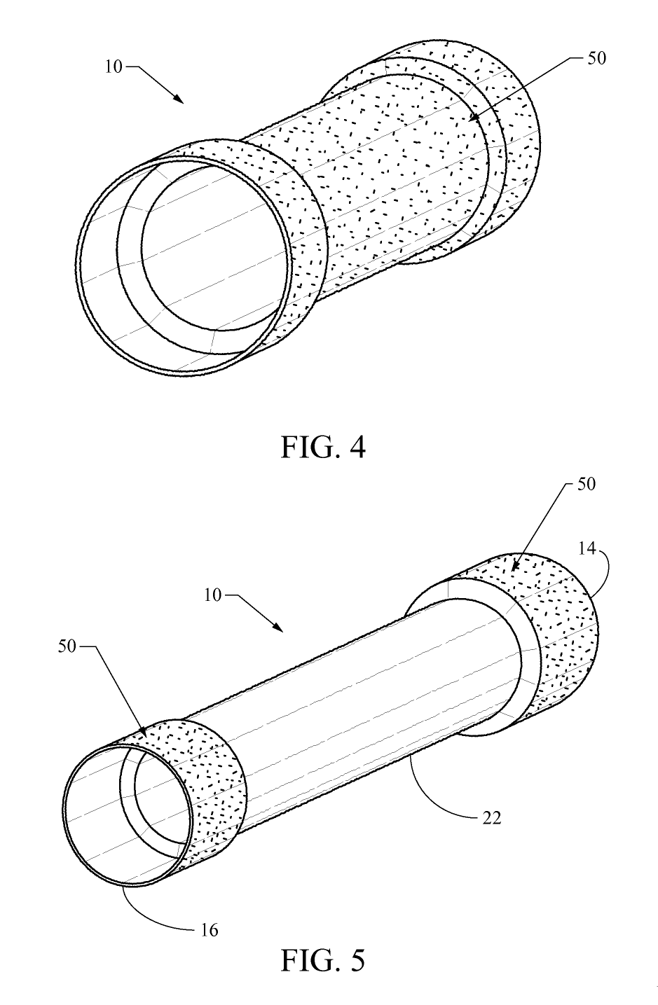

To further reduce the probability of prosthesis migration, at least a portion of the prosthesis 10 is coated with an anti-migration compound. The anti-migration compound may aid in fixing the prosthesis 10 in place relative to the esophagus or any other body vessel into which the prosthesis may be implanted. In one example, the anti-migration compound includes a mucoadhesive agent as further described below. The anti-migration compound may be applied to at least a portion of the outer surface of the prosthesis. FIGS. 4-5 illustrate examples of a prosthesis 10 having an anti-migration compound 50 applied to the outer surface thereof. The prosthesis 10 may be configured generally as described above. The anti-migration compound 50 may be applied to the exterior surface of the sleeve 22, or other covering material, covering the tubular frame of the prosthesis 10. Substantially the entire exterior surface of the sleeve 22 may be coated with the anti-migration compound 50 as shown in FIG. 4. Alternatively a portion of the sleeve 22 may be coated with the anti-migration compound 50. For example, the portions of the sleeve 22 corresponding to the cylindrical end portions 32, 34 of the prosthesis 10 may be coated with the anti-migration compound 50 as shown in FIG. 5. This configuration may aid in fixing the first end 14 and the second end 16 of the prosthesis 10 in place relative to the esophageal wall 90. The anti-migration coating may be applied to the outer surface of the prosthesis 10, or a portion thereof, in any pattern. In one example, the sleeve 22 may be omitted from the prosthesis 10, and the anti-migration coating 50 may be applied to at least a portion of the tubular frame of the prosthesis.

In any of the examples described herein, the prosthesis 10 may be substantially free of coating layers (e.g., between the outer surface of the prosthesis and the anti-migration compound and/or over the anti-migration compound) other than the coating layer that includes the anti-migration compound. In other words, the prosthesis 10 may include a single coating layer that includes the anti-migration compound. This may enable the anti-migration compound to be adhered to each of the outer surface of the prosthesis and the mucous membrane to aid in retaining the prosthesis in place within the body vessel.

The anti-migration compound 50 may be applied to any portion of the prosthesis 10. Preferably, the anti-migration compound 50 may be applied to portions of the prosthesis 10 intended to contact a wall of a body vessel upon implantation of the prosthesis in the body vessel. Upon implantation of the prosthesis 10 within the body vessel, the portions of the prosthesis having the anti-migration compound 50 applied thereto may become fixed or adhered to the wall of the body vessel to reduce the probability of prosthesis migration.

In one example, the anti-migration compound 50 may include any currently known or future developed mucoadhesive agent. The mucoadhesive agent may include any material that is capable of adhering to a mucous membrane which may line the wall of a body vessel or body cavity. The mucous membrane may include a moist mucous layer to which the mucoadhesive agent may adhere. Generally, mucoadhesive agents are hydrophilic macromolecules containing numerous functional groups capable of forming hydrogen bonds. For example, the mucoadhesive agent may include a macromolecule (e.g., a polymer) including repeating monomer units. The mucoadhesive agent may include, for example, a hydrophilic polymer, a hydrogel, a co-polymer, or a thiolated polymer. The hydrogen bond forming functional groups may include carboxyl groups, hydroxyl groups, carbonyl groups, sulphate groups, amide groups, or any other functional groups capable of forming hydrogen bonds. Examples of mucoadhesive agents or components thereof may include, without limitation, carbomers (e.g., polyacrylic acids), polycyclic aromatic hydrocarbons (e.g., retene), carboxylic acids, polyvinylpyrrolidones, polyvinylalcohols, polycarbophils, chitosan materials (i.e., poliglusam, deacetylchitin, or poly-(D)glucosamine), sodium alginates, cellulose derivatives (e.g., methylcellulose, methylethylcellulose, sodium carboxymethylcellulose, hydroxypropylcellulose, hydroxypropylmethylcellulose, or hydroxyethylcellulose), ethers (e.g., polyethylene glycol), lectins (e.g., Erythrina c. lectin, Concanavalin a. lectin, Ulex europaeus lectin, and C-Type lectin), thiamines (e.g., thiamine end capped polymer chains), pathogenic bacteria (e.g., bacterial fimbrins), thiols (e.g., chitosan-cysteine, chitosan-thiobutylamidine, chitosan-thioglycolic acid, polyacrylic acid-cysteine, polyacrylic acid-cysteamine, carboxymethylcellulose-cysteine, or alginate-cysteine), amino acid sequences, ion-exchange resins (e.g., cholestyramine), or any biomolecules including an amino acid sequence (e.g., peptides). Other examples of mucoadhesive agents or components thereof may include guar gum, karya gum, xantham gum, locust bean gum, acacia gum, gellan gum, tragacanth, soluble starch, gelatin, or pectin. In some examples, mucoadhesive agents may include any biomolecules having an affinity for a mucosa such as, for example, proteins (e.g., fimbrial proteins or affinity ligands).

A mucoadhesive agent may adhere to a mucous membrane by physical and/or chemical forces including, for example, ionic bonding, covalent bonding, hydrogen bonding, Van-der-Waals bonding, or hydrophobic bonding (i.e., hydrophobic interaction).

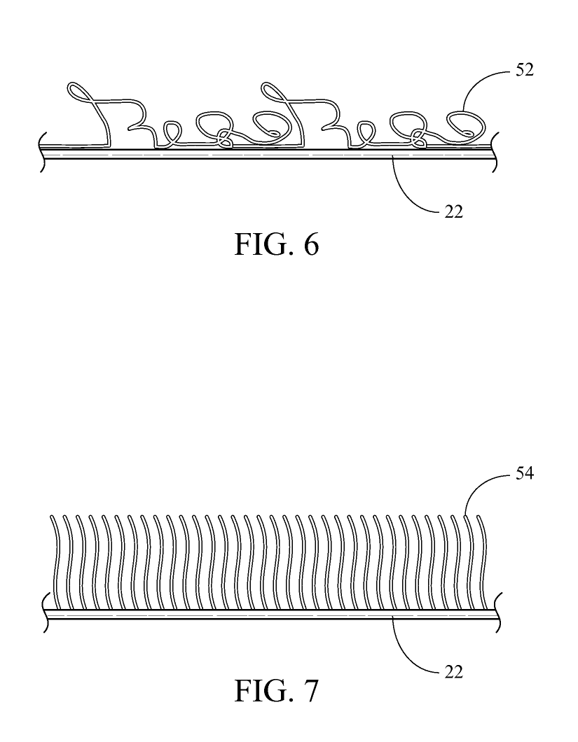

The anti-migration compound 50 may be applied to the prosthesis 10 using any suitable technique. For example, the anti-migration compound 50 may be applied by dipping the prosthesis 10, or a portion thereof, into a solution containing the mucoadhesive agent. In another example, the anti-migration compound 50 may be sprayed onto the prosthesis 10. The mucoadhesive agent may be combined with a suitable solvent and/or other components of the anti-migration compound 50 prior to dipping or spraying the prosthesis 10. After application of the solution or suspension including the anti-migration compound 50 to the prosthesis 10, the solvent may be evaporated to form a coating of the anti-migration compound on the exterior surface of the prosthesis 10. The coating may include a plurality of polymer chains 52, including the mucoadhesive agent, disposed on the surface of the prosthesis 10 as shown in FIG. 6. The polymer chains 52 may be disposed on the surface of the prosthesis 10 in a substantially unstructured or random arrangement. The unstructured arrangement may be formed during the dipping or spraying process used to coat the prosthesis 10 with the anti-migration compound 50.

In one example, the anti-migration compound 50 may be covalently bonded to the exterior surface of the prosthesis 10. FIG. 7 shows one example of a plurality of polymer chains 54, including the mucoadhesive agent, covalently bonded to the prosthesis 10. In this example, the sleeve 22, or other covering material, of the prosthesis 10 may be formed from a polymer material. The sleeve 22 may be formed from any suitable polymer material such as, for example, silicone, polyurethane, polyethylene, or polyamide (e.g., nylon). The exterior surface of the sleeve 22 may be activated by any suitable surface activation technique. For example, the polymer material may be activated by exposing the exterior surface of the sleeve 22 to ozone, plasma, and/or ultraviolet radiation. Additionally, or alternatively, the polymer material may be activated by removal of a halogen which may be present on the exterior surface of the polymer material. Such surface activation may promote the formation of polar functional groups on the exterior surface of the sleeve 22 to promote the adhesion of the components of the anti-migration compound 50 to the prosthesis 10.

The polymer chains of the anti-migration compound 50, including the mucoadhesive agent, may be formed on the activated surface of the polymer material using any suitable polymerization method. For example, the mucoadhesive agent may be formed on the activated surface by free radical polymerization. The polymer chain that forms the mucoadhesive agent may be formed by bonding a first free radical monomer unit to the activated surface of the polymer material and then successively adding additional free radical monomer units to form the desired polymer chain. By adding the desired monomer units, the mucoadhesive polymer may be grown from the activated surface of the sleeve 22. The process of growing a polymer chain from an activated surface by successively adding monomer units may be referred to as a “grafting from” process.

In another example, the polymer chain may be formed using any suitable method and the formed polymer chain may be covalently bonded to the polymer material of the sleeve 22. Such a process may be referred to as a “grafting to” process. A grafting to process may include one or more of plasma exposure, ultraviolet irradiation, atom transfer radical polymerization (ATRP), reversible addition fragmentation chain transfer (RAFT) polymerization, and removal of a halogen present on the exterior surface of the polymer material and radical polymerization. A grafting to process may be more difficult to perform than a grafting from process because it may be more difficult to bond relatively long polymer chains to the activated site of the polymer material than to bond relatively short monomer units to the activated site of the polymer material.

Regardless of the grafting process used, the resulting polymer chains 54 may be bonded to and extend from the activated polymer material surface of the prosthesis 10. The polymer chains 54 may include any of the functional groups described above to impart mucoadhesive properties thereto. In this manner, the mucoadhesive agent may be covalently bonded to the surface of the prosthesis 10 to create a coating of mucoadhesive agent on the exterior surface of the prosthesis. Such covalent bonding may inhibit the mucoadhesive agent from separating from the prosthesis 10 (e.g., by flaking off of the prosthesis) and, thus, may strengthen the adhesion between the prosthesis and the mucous membrane after implantation of the prosthesis within the body vessel.

Polymerizing the mucoadhesive agent on the activated surface of the sleeve 22 may form a series of polymer chains 54 covalently bonded to and extending from the surface of the sleeve. Entanglement and/or crosslinking between the individual polymer chains 54 may be reduced, as compared to a polymer coating that is applied by dipping or spraying, so that the individual polymer chains may be arranged as a plurality of substantially separate strands as shown in FIG. 7. This structured arrangement may be referred to as a “brush formation” in which the polymer chains 54 of the mucoadhesive agent are covalently attached to the sleeve 22. The amount of crosslinking between the individual polymer chains 54 may be inversely proportional to the strength of the adhesion of the mucoadhesive agent to the mucous membrane (i.e., mucoadhesion binding). Such a structured arrangement and/or reduced crosslinking of the polymer chains 54 may enable increased interaction between the functional units of the mucoadhesive agent and the mucous membrane, which may enhance the strength of the adhesion of the mucoadhesive agent to the mucous membrane. In other words, this configuration may enable a greater degree of interaction or bonding between the polymer chains 54 extending from the exterior surface of the prosthesis 10 and the mucous membrane of the body vessel. Such increased interaction or bonding may provide an increased adhesive force between the prosthesis 10 and the mucous membrane to prevent migration of the prosthesis within the body vessel. In one example, cross-link density (e.g., between the polymer chains 54) may be kept to a minimum. To that end, the polymer chains of the anti-migration coating may be substantially uncrosslinked. Mucoadhesion of crosslinked polymers may be increased by the incorporation of free polymer chains or polymer grafted into the crosslinked network.

In any of the examples described herein, the mucoadhesive agent of the anti-migration compound 50 may be configured to adhere to a mucous membrane lining the wall of a body vessel. In other words, the properties of the anti-migration compound 50 and/or the mucoadhesive agent may be configured to achieve a desired adhesive force between the mucoadhesive agent and the mucous membrane. In one example, the thickness of the coating of the anti-migration compound 50 on the prosthesis 10 may affect the affinity of the mucoadhesive agent for the mucous membrane. The thickness of the coating of the anti-migration compound 50 may be a function of the length of the polymer chains of the mucoadhesive agent. In other words, the length of the polymer chains of the mucoadhesive agent may affect the affinity of the mucoadhesive agent for the mucous membrane. For example, the thickness of the coating of the anti-migration compound 50 and/or the length of the polymer chains of the mucoadhesive agent may be greater than or equal to about 2 μm, preferably greater than or equal to about 4 μm in the dry state. Upon hydration of the anti-migration compound, the thickness and/or the length may increase (e.g., by swelling). For example, the thickness and/or length may increase to greater than or equal to about 8 μm. In other examples, the coating of the anti-migration compound 50 may have any suitable thickness, and the polymer chains of the mucoadhesive agent may have any suitable length.

Additionally, or alternatively, the molecular weight of the mucoadhesive agent may affect the affinity of the mucoadhesive agent for the mucous membrane. For example, the molecular weight of the mucoadhesive agent may be less than or equal to about 200,000, typically less than or equal to about 150,000, preferably less than or equal to about 100,000. In other examples, the molecular weight of the mucoadhesive agent may have any suitable molecular weight.

Additionally, or alternatively, the penetration depth of the polymer chains of the mucoadhesive agent may affect the affinity of the mucoadhesive agent for the mucous membrane. In other words, the depth of the penetration of the polymer chains of the mucoadhesive agent into the mucous membrane and/or the tissue of the body vessel may affect the strength of the adhesive force between the mucoadhesive agent and the body vessel (i.e., the strength of the bioadhesive bond). For example, upon implantation of the prosthesis 10 in the body vessel, the polymer chains of the mucoadhesive agent may penetrate the mucous membrane and/or the tissue to a depth of from about 0.2 μm to about 0.5 μm. In other examples, the polymer chains of the mucoadhesive agent may penetrate the mucous membrane and/or the tissue to any suitable depth.

The anti-migration coating 50 may be applied to the surface of any conventional, commercially available prosthesis. For example, a conventional GI stent (e.g., a bare, covered, or partially covered stent) may be coated with a mucoadhesive agent as described above. Also for example, the silicone membrane of a conventional GI stent may be activated, as described above, to enable covalent bonding of a mucoadhesive agent to the silicone membrane.

In one example, the anti-migration compound 50 may be configured as a biodegradable compound. Such a biodegradable compound may degrade at any desired rate upon implantation within a body vessel. The biodegradable anti-migration compound 50 may fix the prosthesis 10 to the wall of the body vessel upon implantation of the prosthesis and then degrade over time to release the prosthesis from the wall of the body vessel. This may enable removal of the prosthesis from the body vessel. Alternatively, or additionally, the prosthesis 10 may be formed from one or more biodegradable materials. Thus, after implantation of the prosthesis 10, the prosthesis and the anti-migration compound 50 may degrade over time. Such a prosthesis may not require intervention to remove the prosthesis from the body because the prosthesis may degrade within the body.

Biodegradable anti-migration compounds and/or biodegradable prostheses may be particularly well-suited for use in treating benign tumors. In treating a benign tumor, a physician may implant a prosthesis within the diseased body vessel for a relatively short period of time. Thus, a biodegradable anti-migration compound may enable the physician to remove the prosthesis after treatment of the tumor. A biodegradable prosthesis may degrade within the body such that the physician may choose not to remove the prosthesis, but rather allow the prosthesis to degrade within the body.

The anti-migration coating 50 may include a bioactive ingredient. Inclusion of a bioactive ingredient in the anti-migration coating 50 may promote localized or site specific delivery of the bioactive ingredient to the body vessel in which the prosthesis 10 is implanted. The bioactive ingredient may be provided in any suitable form including, for example, a pharmaceutically acceptable salt, a prodrug, or a derivative or analog of a compound named herein, or equivalents thereto. Any suitable bioactive ingredient may be used including, for example, proteins, affinity ligands, plasmid DNA, cells, lipids, amino acid based biomolecules, or cell adhesion molecules (CMAs).

While certain preferred embodiments relating to a coated esophageal prosthesis are described herein, the coatings may be applied to any implantable medical device. The coated medical device may be any device that is adapted for introduction temporarily or permanently into the body for the prophylaxis or therapy of a medical condition. For example, such medical devices may include, but are not limited to, stents, stent grafts, vascular grafts, catheters, guide wires, balloons, filters (e.g. vena cava filters), cerebral aneurysm filler coils, sutures, staples, anastomosis devices, vertebral disks, bone pins, suture anchors, hemostatic barriers, clamps, screws, plates, clips, slings, vascular implants, tissue adhesives and sealants, tissue scaffolds, myocardial plugs, pacemaker leads, valves (e.g. venous valves), abdominal aortic aneurysm (AAA) grafts, embolic coils, various types of dressings, bone substitutes, intraluminal devices, vascular supports, or other known bio-compatible devices.

In general, an endoluminal prosthesis typically includes a tubular frame formed from a plurality of interconnected struts and bends defining apertures or open spaces therebetween. The struts and bends may form a plurality of sinusoidal hoop members longitudinally aligned to form a cylindrical structure. A tubular frame may have other configurations, such as braided tubes or interconnected helical flexible members. Typical structures include: an open-mesh network including one or more knitted, woven or braided metallic filaments; an interconnected network of articulable segments; a coiled or helical structure including one or more metallic filaments; and a patterned tubular metallic sheet (e.g., a laser cut tube). The tubular frame may be covered by any suitable biocompatible material to form a covered prosthesis. Examples of endoluminal prostheses include endovascular, biliary, tracheal, gastrointestinal, urethral, ureteral, esophageal and coronary prostheses.

In one embodiment, the prosthesis may be configured as a self-expanding or balloon-expandable device. The device may be a bifurcated stent, a coronary vascular support frame, a urethral stent, a ureteral stent, a biliary stent, a tracheal stent, a gastrointestinal stent, or an esophageal stent, for example. The tubular frame of the prosthesis may be made of one or more suitable biocompatible materials such as stainless steel, nitinol, NP35N, gold, tantalum, platinum or platinum irdium, niobium, tungsten, iconel, ceramic, nickel, titanium, stainless steel/titanium composite, cobalt, chromium, cobalt/chromium alloys, magnesium, aluminum, or other biocompatible metals and/or composites or alloys.

It is intended that the foregoing detailed description of medical devices and methods be regarded as illustrative rather than limiting, and that it be understood that it is the following claims, including all equivalents, that are intended to define the spirit and scope of this invention. Terms are to be given their reasonable plain and ordinary meaning. Also, the embodiment of any figure and features thereof may be combined with the embodiments depicted in other figures. Other features known in the art and not inconsistent with the structure and function of the present invention may be added to the embodiments.

Drawings in the figures illustrating various embodiments are not necessarily to scale. Some drawings may have certain details magnified for emphasis, and any different numbers or proportions of parts should not be read as limiting, unless so-designated in the present disclosure. Those skilled in the art will appreciate that embodiments not expressly illustrated herein may be practiced within the scope of the present invention, including those features described herein for different embodiments, and may be combined with each other and/or with currently-known or future-developed technologies while remaining within the scope of the claims presented here. Moreover, the advantages described herein are not necessarily the only advantages of the invention and it is not necessarily expected that every embodiment of the invention will achieve all of the advantages described. It is therefore intended that the foregoing detailed description be regarded as illustrative rather than limiting. And, it should be understood that the following claims, including all equivalents, are intended to define the spirit and scope of this invention.

We claim:

1. A prosthesis for placement within a body vessel of a patient, the prosthesis having an outer surface and comprising:
a tubular frame;
a covering material coupled to the tubular frame, the covering material having an outer surface defining at least a segment of the outer surface of the prosthesis; and
an anti-migration coating applied to at least a portion of the outer surface of the prosthesis, the anti-migration coating comprising a mucoadhesive agent, the mucoadhesive agent comprising a polymer configured to adhere to a mucous membrane within the body vessel, the anti-migration coating comprising a thickness of greater than or equal to about 2 μm in a dry state to promote adhesion to the mucous membrane.

2. The prosthesis of claim 1, wherein the anti-migration coating is applied to substantially the entire outer surface of the prosthesis.

3. The prosthesis of claim 1, wherein the outer surface of the prosthesis comprises a first contact area positioned near a proximal end of the prosthesis and a second contact area positioned near a distal end of the prosthesis, and the anti-migration coating is applied to at least a portion of the first contact area and at least a portion of the second contact area.

4. The prosthesis of claim 3, wherein the tubular frame has a compressed configuration and an expanded configuration, and with the tubular frame in the expanded configuration within the body vessel, at least a portion of each of the first contact area and the second contact area engages a wall of the body vessel to retain the prosthesis in place relative to the body vessel.

5. The prosthesis of claim 4, wherein with the tubular frame in the expanded configuration, each of the proximal end and the distal end of the prosthesis has a diameter that is greater than a diameter of an intermediate segment of the prosthesis.

6. The prosthesis of claim 1, wherein the mucoadhesive agent comprises at least one material selected from the group consisting of sodium carboxymethyl cellulose, a lectin, a xantham gum, a thiamine, and any combination thereof.

7. The prosthesis of claim 1, wherein the mucoadhesive agent comprises a molecular weight of less than or equal to about 200,000.

8. The prosthesis of claim 1, wherein the anti-migration coating further comprises a bioactive ingredient.

9. The prosthesis of claim 1, wherein the tubular frame comprises a plurality of stents.

10. The prosthesis of claim 1, wherein the tubular frame comprises a closed cell structure.

11. The prosthesis of claim 1, wherein the mucoadhesive agent comprises a plurality of polymer chains disposed on the outer surface of the prosthesis and comprising an unstructured arrangement.

12. The prosthesis of claim 1, wherein the mucoadhesive agent comprises a plurality of polymer chains disposed on the outer surface of the prosthesis and covalently bonded to the outer surface of the prosthesis.

13. The prosthesis of claim 12, wherein the plurality of polymer chains comprises a structured arrangement.

14. The prosthesis of claim 1, wherein the covering material comprises a tubular sleeve.

15. A method for making a prosthesis for placement within a body vessel of a patient, the method comprising:
providing a tubular frame;
coupling a covering material to the tubular frame, the covering material having an outer surface;
applying an anti-migration coating comprising a thickness of greater than or equal to about 2 μm in a dry state to at least a portion of the outer surface of the covering material, the anti-migration coating comprising a mucoadhesive agent, the mucoadhesive agent comprising a polymer configured to adhere to a mucous membrane within the body vessel.

16. The method of claim 15, wherein the applying step comprises covalently bonding at least one monomer unit of the mucoadhesive agent to the outer surface of the covering material.

17. The method of claim 16, wherein the covering material comprises a polymer material, and the applying step comprises activating the outer surface of the covering material by exposing the outer surface of the covering material to at least one of ozone, plasma, or ultraviolet radiation.

18. A method of treating an esophagus, the method comprising:
introducing an expandable prosthesis into the esophagus in a compressed configuration;
expanding the prosthesis from the compressed configuration such that the prosthesis engages a wall of the esophagus, the prosthesis comprising a mucoadhesive agent disposed on an outer surface of prosthesis, the mucoadhesive agent comprising a polymer configured to adhere to a mucous membrane disposed on the wall of the esophagus; and
adhering the prosthesis to the mucous membrane of the esophagus with the mucoadhesive agent to fix the prosthesis in place relative to the esophagus.

19. The method of claim 18, wherein the polymer of the mucoadhesive agent comprises a molecular weight of less than or equal to about 200,000.

20. The method of claim 18, further comprising penetrating the mucous membrane with the polymer of the mucoadhesive agent to a depth of about 0.2 μm to about 0.5 μm.
